(19) United States
(12) Patent Application Publication
(10) Pub. No.: US 2002/0111680 A1
(43) Pub. Date: Aug. 15, 2002

(54) Ratcheted bone dowel

(76) Inventor: Gary K. Michelson, Venice, CA (US)

Correspondence Address: MARTIN & FERRARO LLP, Suite 300, 14500 Avion Parkway, Chantilly, VA, 20151-1101, US

(21) Appl. No.: 09/991,247

(22) Filed: Nov. 15, 2001

Related U.S. Application Data
(63) Continuation-in-part of application No. 09/593,591, filed on Jun. 13, 2000.
(60) Provisional application No. 60/249,802, filed on Nov. 16, 2000.

(51) Int. Cl. A61F 2/44

(52) U.S. Cl. 623/17.11

(57) ABSTRACT

An interbody spinal implant for insertion at least in part into an implantation space formed across a disc space between adjacent vertebral bodies of a human spine and into at least a portion of the endplates of the vertebral bodies. The implant includes a body having a leading end for insertion first into the disc space and a trailing end opposite the leading end and opposite upper and lower surfaces adapted to be placed in contact with and to support the adjacent vertebral bodies; the upper and lower surfaces being arcuate. The implant also has an opening passing through the upper and lower surfaces for permitting for the growth of bone from adjacent vertebral body to adjacent vertebral body through the implant. The implant is manufactured from a composite of cortical bone and at least one bioresorbable material. The cortical bone and at least one bioresorbable material being combined to form a machinable material from which the implant is manufactured.

### BACKGROUND OF THE INVENTION

This application claims the benefit of provisional application Ser. No. 60/249,802, filed Nov. 17, 2000, and is a continuation of application Ser. No. 09/593,591, filed Jun. 13, 2000, both of which are incorporated herein by reference.

### FIELD OF THE INVENTION

The present invention relates to bone dowels to be placed across the intervertebral space left after the removal of a damaged spinal disc.

### DESCRIPTION OF RELATED ART

In the past, Cloward, Wilterberger, Crock, Vich, Bagby, Michelson and others have taught various methods involving the drilling of holes across the disc space between two adjacent vertebral bodies of the spine for the purpose of causing an interbody spinal fusion. Cloward taught placing a dowel of bone within that drilled hole for the purpose of bridging the defect and to be incorporated into the fusion. Vich taught the threading of that bone dowel. Bagby taught the placing of the bone graft into a metal bucket otherwise smooth on its surface, except for rows of radially placed holes communicative to the interior of the basket and to the bone graft. The Bagby device was disclosed as capable of being used in a horse.

Several problems exist in the prior art in that threaded bone dowels are often subject to a potentially disruptive torquing force that can damage the bone dowels and a motion that puts the surrounding tissues at risk of being wound up and torn. To accommodate the torque associated with the insertion of threaded bone dowels, the walls of the bone dowels must be sufficiently thicker, thereby decreasing the available storage area for fusion enhancing substances.

Another problem can arise when placing two cylindrical bone dowels side-by-side across a disc space and into two adjacent vertebral bodies. Two cylindrical bone dowels are considered to be the preferred number of dowels versus one for a more stable construct due to increasing the surface area and so as to prevent rocking in comparison to a single bone dowel placed centrally. Where the height of the disc space requires a bone dowel having a sufficiently large diameter to penetrate into and significantly engage each of the adjacent vertebral bodies, it is not possible to place two such bone dowels side-by-side and contain them within the transverse width of the spine. If one were to use smaller diameter bone dowels placed side-by-side sized to fit within the transverse width of the spine, then the bone dowels would have an insufficient height to adequately engage the bone. Abandoning the side-by-side double bone dowel construct in favor of a single, centrally placed bone dowel, would require utilizing a bone dowel sufficiently large enough to occupy a sufficient portion of the transverse width of the disc space to promote firm stability. The vertical height and excursion into the adjacent vertebral bodies of such a centrally placed bone dowel would be so severe that if any two consecutive disc spaces were to be operated upon, the vertebral body in between would be cut in half.

With non-threaded, smooth-surfaced bone dowels, the lack of any structure to keep the bone dowels secured once inserted can lead to the undesirable and dangerous expulsion of the bone dowels from the patient.

Artificially created implants have been used in an attempt to solve the above problems and have met with varying degrees of success. However, artificial implants do not allow bone to biologically participate in the fusion process to the extent that a bone dowel does.

There is therefore the need for a bone dowel that is capable of being fully inserted into the spine at least by linear advancement and in certain embodiments subsequent rotation and yet possesses structure for retaining the bone dowel once implanted.

### SUMMARY OF THE INVENTION

The various embodiments of the bone dowels of the present invention all have in common a substantially cortical structure which may have a passageway through the bone dowel in communication between opposed upper and lower arcuate surfaces of the bone dowel adapted to penetrably engage the adjacent vertebral bodies. The bone dowel may be filled with fusion promoting substances including, for example, cancellous bone, hydroxyapatite, hydroxyapatite tricalcium phosphate, genes coding for the production of bone, or bone morphogenetic protein.

The opposed arcuate surfaces may be generally parallel over the bone dowel length, convergent, or divergent, or any combination thereof.

All of the bone dowels of the present invention preferably have a plurality of at least partially circumferential ratchetings along at least a substantial portion of the opposed surfaces, a leading end, and a trailing end opposite the leading end. The trailing end is preferably adapted to cooperatively engage a driver, however, the bone dowels alternatively may be impacted into position by a (mechanical) non-engaging driver.

The bone dowels of the present invention may be formed by cutting diametrically across the diaphyseal portions of a human long bone such as those found in the extremities, and particularly the larger bones such as the femur, tibia, and humerus.

Alternatively, the bone dowels of the present invention would anticipate and still include a composite of cortical bone and a second material, which need not, but preferably would be bioresorbable, to form a machineable or moldable material from which interbody bone dowels might be formed. Such bone dowels may have a passageway or hollow portion from a first vertebral body engaging surface to an opposed second vertebral body engaging surface for loading with fusion promoting substances. Alternatively, fusion promoting substances and a passageway may be omitted. In this event, the bone dowel itself made at least in part of bone promotes the fusion process.

Bone dowels of the present invention are far stronger than the classic cortico-cancellous bone grafts of Cloward, which included a column of cancellous bone sandwiched between two end discs of cortical bone.

Bone dowels of the present invention are inserted into sites prepared across the height of the disc space having resected arcs of bone through the opposed vertebral endplates. The insertion site may be achieved by drilling generally parallel across the height of the disc space with a drill or mill having an outer diameter greater than the restored disc space height as desired by the surgeon.

Certain of the present invention embodiments may be “locked into position” once already fully linearly inserted by rotating them generally 90 degrees about their longitudinal axis.

The bone dowels of the present invention come in various basic forms. In one embodiment, the bone dowel of the present invention has a fully circumferential body and fully circumferential ratchets.

In another embodiment, the bone dowel of the present invention has at least one side with ratchets tangentially cut off and preferably both opposed sides cutoff. The cutoff areas are preferably flat and parallel.

In an additional embodiment, the bone dowel of the present invention has a fully round minor root diameter with the sides cut off of the ratchets.

The above-described embodiments of the present invention can, inter-alia, include the following variations:

1) they all can be generally cylindrical; and

2) they may have convergent upper and lower surfaces to the body.

If the bone dowels are to be used for posterior lumbar interbody fusion, then they may be constructed with or without flat smooth sides.

The bone dowels of the present invention may be adapted to receive opposed vertebral body engaging screws of cortical bone, bioresorbable materials, and other materials through their trailing ends.

The bone dowels of the present invention can be configured to have side walls that have a complementary combined width less than a combined height. The present inventive bone dowels may be adapted for side-by-side contact placement wherein one or more of the sides in contact are flat or configured to cooperatively engage the side of the other bone dowel with which it is in contact. For example, a second bone dowel may be C-shaped in transverse cross-section such that the opened end of the C-shape is oriented toward the other of the bone dowels when implanted. Thus, it is possible to place two such bone dowels side-by-side across a disc space and into two adjacent vertebral bodies in close approximation to each other and within the transverse width of the spine, where the transverse width of the spine would have otherwise been insufficient relative to the required bone dowel height to have allowed for the accommodation of two prior art cylindrical threaded bone dowels.

### BRIEF DESCRIPTION OF THE DRAWINGS

FIG. 1 is a diagrammatic representation of a segment of the human spinal column comprising several vertebral bodies with various bone dowels inserted across the disc space and into the two adjacent vertebral bodies to illustrate the problems encountered by those bone dowels;

FIG. 2 is a front elevation view of a long bone from which a bone dowel has been cut;

FIG. 3 is an exploded front view of the area along line 3-3 of FIG. 2;

FIG. 4 is a top plan view of an embodiment of the bone dowel of the present invention having surface roughenings in the form of ratchetings;

FIG. 5 is a side elevation view of the bone dowel of FIG. 4;

FIG. 6 is a trailing end elevation view of the bone dowel of FIG. 4;

FIG. 7 is a top plan view of an alternative embodiment of the bone dowel of the present invention;

FIG. 8 is a side elevation view of the bone dowel of FIG. 7;

FIG. 9 is a trailing end elevation view of the bone dowel of FIG. 7;

FIG. 9A is an alternative trailing end elevation view of the bone dowel of FIG. 7;

FIG. 10 is a top plan view of an alternative embodiment of the bone dowel of the present invention;

FIG. 11 is a side elevation view of the bone dowel of FIG. 10;

FIG. 12 is a trailing end elevation view of the bone dowel of FIG. 10;

FIG. 13 is a top plan view of an alternative embodiment of the bone dowel of the present invention;

FIG. 14 is a side elevation view of the bone dowel of FIG. 13;

FIG. 15 is a trailing end elevation view of the bone dowel of FIG. 13;

FIG. 16 is a top plan view of yet another embodiment of the bone dowel of the present invention;

FIG. 17 is a side elevation view of the bone dowel of FIG. 16;

FIG. 18 is a trailing end elevation view of the bone dowel of FIG. 16;

FIG. 19 is a diagrammatic representation of a segment of the human spinal column showing a bone dowel of FIG. 9A of the present invention inserted within the spine;

FIG. 20 is a diagrammatic representation of a segment of the human spinal column showing a bone dowel of FIG. 19 inserted within the spine and rotated approximately 90 degrees; and

FIG. 21 is an elevational side view of a segment of the spinal column with an alternative embodiment of two bone dowels of the present invention having corresponding concave and convex sides inserted across one disc space.

### DETAILED DESCRIPTION OF THE INVENTION

Reference will now be made in detail to the present preferred embodiments of this invention, examples of which are illustrated in the accompanying drawings. Similar reference numbers such as “102, 202” will be used throughout the drawings to refer to similar portions of different embodiments of the present invention.

The Previous Devices

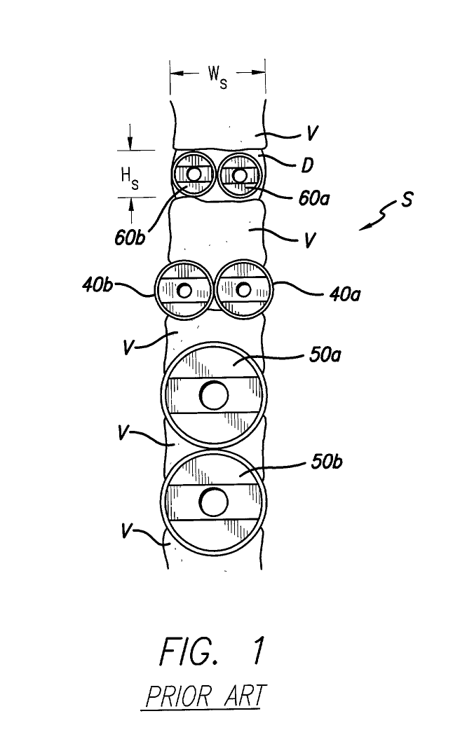

As shown in FIG. 1, a diagrammatic representation of a segment of the human spinal column generally referred to by the letter S is shown. The segment of the spinal column S comprises several vertebral bodies V and a disc space D between two adjacent vertebral bodies V. Various cylindrical threaded bone dowels, each having different diameters, are shown inserted across disc space D.

When the height Hₛ of disc space D is so large that two cylindrical bone dowels, such as bone dowels 40a, 40b, each having a sufficient diameter to cross disc space D and sufficiently engage into the bone of adjacent vertebral bodies V, are placed across disc space D, the combined overall width of bone dowels 40a and 40b exceeds the transverse width Wₛ of spinal column S. As a result, a portion of each implant 40a, 40b protrudes from the sides of spinal column S and could cause severe and perhaps mortal damage to the patient as delicate and vital structures lie adjacent to that area of spinal column S such that the use of two cylindrical bone dowels 40a, 40b would not be desirable.

If instead of two bone dowels 40a, 40b, a single dowel, such as bone dowel 50a, were to be used having a sufficient diameter to provide for stability and fusion, then bone dowel 50a would penetrate deeply into the adjacent vertebral bodies V. Bone dowel 50a would have a diameter that is significantly greater than height Hₛ of disc space D, such that vertebral bodies V would have to be substantially bored out to accommodate the large diameter of bone dowel 50a. As a result, a large part of vertebral bodies V would be removed, and thus the overall structural integrity of vertebral bodies V would be substantially weakened. This is especially a problem when a second bone dowel 50b identical to bone dowel 50a is placed across disc space D on the other side of the same vertebral body V such that two bone dowels 50a, 50b are placed across disc spaces D on either side of vertebral body V. As a result, vertebra V is cleaved into a “butterfly” configuration as shown in FIG. 1, and the structural integrity and strength of vertebral bodies V is further diminished such that the effectiveness of the spinal fusion process is substantially reduced, and vertebral bodies V are at risk of devascularization and fracture.

Conversely, if two cylindrical bone dowels such as bone dowels 60a, 60b, each having a sufficiently sized diameter such that when placed side-by-side in disc space D, the combined overall width of bone dowels 60a, 60b just fills transverse width Wₛ of spinal column S, the diameter of each of bone dowels 60a, 60b will not be sufficient to cross disc space D to engage vertebral bodies V. Therefore, while the bone dowels 60a, 60b will not protrude from the sides of spinal column S, bone dowels 60a, 60b cannot reach and engage the bone of vertebral bodies V and thus cannot function to stabilize adjacent vertebral bodies V.

The present invention

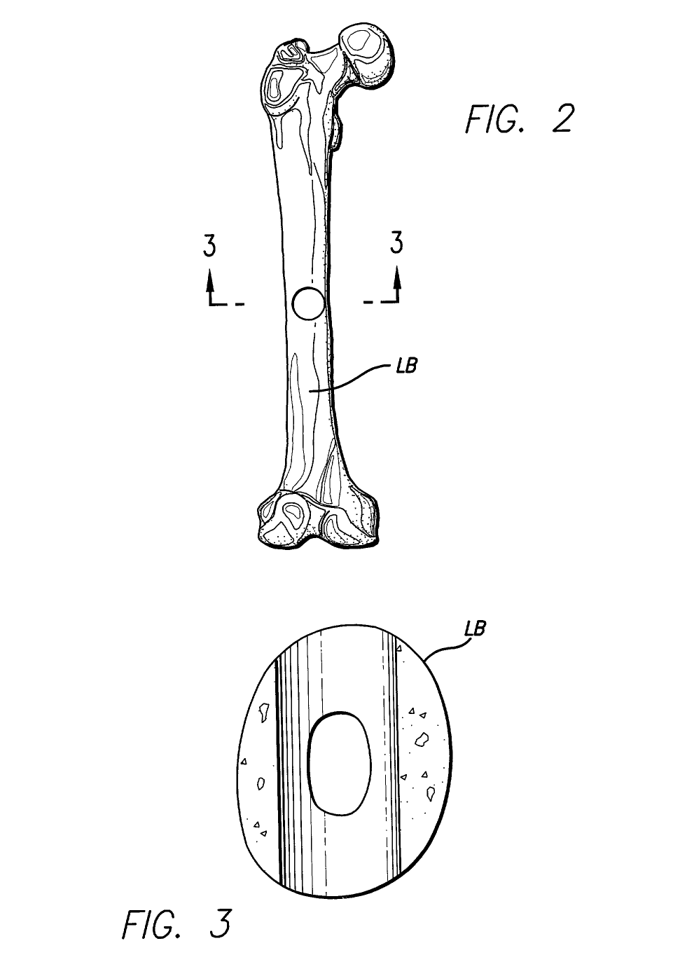

The present invention is directed to a bone dowel preferably composed substantially of cortical bone. As shown in FIGS. 2 and 3, the bone dowel may be formed by cutting diametrically across the diaphyseal portions of a mammalian long bone LB, such as those found in the extremities, e.g., the femur, tibia, and humerus. An alternative method of forming a bone dowel of the present invention is to create a composite of cortical bone including fibers, filaments, and/or particles, and one or more materials that need not, but preferably would be bioresorbable, such as plastic, ceramic, and/or composite plastic, to form a machineable material from which the bone dowel might be formed.

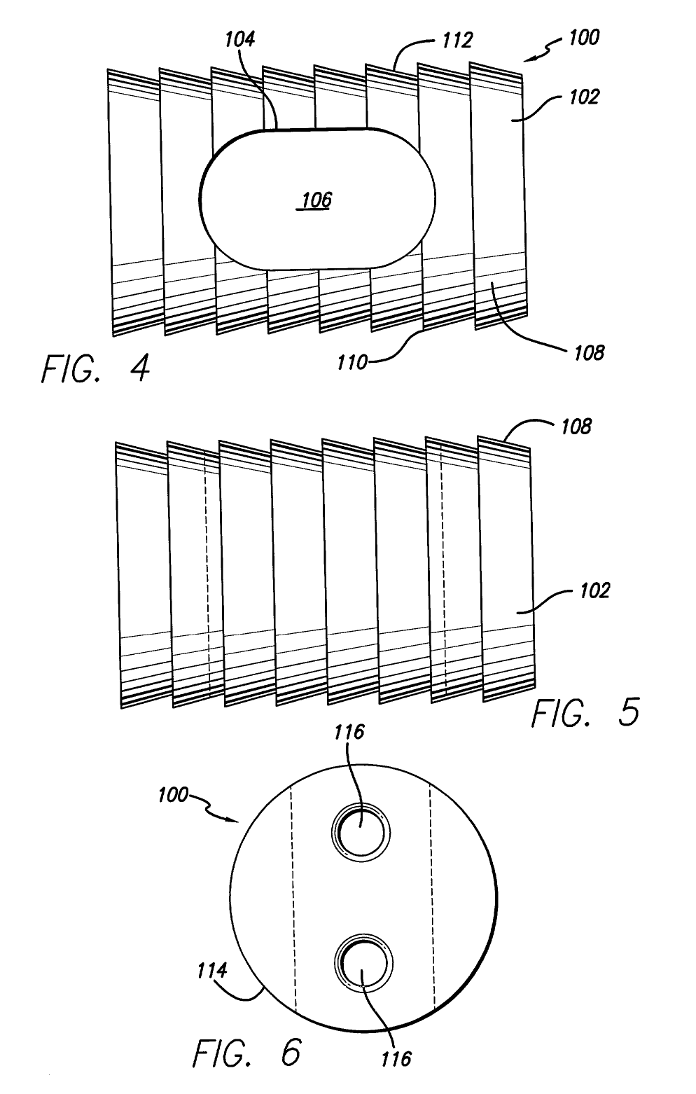

As shown in FIGS. 4-6, a preferred embodiment of the bone dowel of the present invention is shown and generally referred to by the numeral 100. Bone dowel 100 preferably has a substantially cylindrical configuration having an arcuate outer wall 102 having opposed openings 104 leading to a passageway 106. The exterior of bone dowel 100 comprises surface roughenings, preferably ratchetings 108 that provide a surface suitable for engaging adjacent vertebral bodies to stabilize bone dowel 100 across the disc space and into the adjacent vertebral bodies once surgically implanted. In FIGS. 4-6, ratchetings 108 extend around the circumference of bone dowel 100. Each of the ratchetings 108 has a bone-engaging edge 110 and an angled segment 112. Ratchetings 108 are preferably forward facing with angled segment 112 facing the direction of insertion for a one-way insertion of the bone dowel.

Each of the ratchetings 108 has a height that is substantially less than the height of a requisite thread for a cylindrical threaded bone dowel of the same size. As a thread is a simple device for converting torque to linear advancement, the requisite height of the thread is proportional to the surface area and diameter of the bone dowel and must be sufficient to pull a cylindrical bone dowel having a diameter sufficient to cross the disc space through a material as dense as bone. In contrast, ratchetings 108 have a height that is significantly less than the requisite height of a thread of a same-sized, threaded bone dowel since bone dowel 100 is implanted across the disc space and into each adjacent vertebral body by linear advancement. Bone dowel 100 may be pushed into the disc space by direct linear advancement since it requires no thread to pull it forward through the spine. As no torque is required to advance bone dowel 100, there is no minimum requisite height of the surface roughenings. The preferred surface feature gives bone dowel 100 stability once implanted.

Ratchetings 108 preferably face in one direction, the direction in which bone dowel 100 is inserted, and function to prevent bone dowel 100 from backing out of the disc space in a direction opposite to the direction of insertion once inserted between the two adjacent vertebral bodies. Ratchetings 108 urge bone dowel 100 forward against the unremoved bone of the vertebral bodies. To the extent that bone dowels move, they generally back out along the same path in which they are inserted. Repeated movement of a patient's body over time may cause some other design of bone dowel to come loose. Ratchetings 108 of the present invention tend to urge bone dowel 100 forward against the solid unremoved bone further resisting dislodgement and controlling motion, resulting in an exceedingly stable implantation.

Bone engaging edges 110 of ratchetings 108 have a height at a highest point measured from the root diameter of bone dowel 100 that is approximately 0.35 mm. In this manner, bone dowel 100 may be placed beside a second of its kind at a distance of approximately 0.7 mm apart, or, if offset, even closer, substantially reducing the combined overall width of two bone dowels 100 once surgically implanted. Ratchetings 108 may have a height in the range of 0.25-1.5 mm, with the preferred height range being 0.35-0.75 mm.

The decreased combined overall width of two bone dowels 100 is the difference between the root and major diameters of each bone dowel 100 and is achieved by utilizing surface roughenings such as ratchetings 108 for stability. The surface roughenings allow the two bone dowels to come into considerably closer approximation to one another and require less total transverse width for their insertion than is possible for two threaded cylindrical bone dowels having identical root diameters because of the requisite thread height of such threaded bone dowels. Reducing the offset between bone dowels allows for the use of larger diameter bone dowels which can then still fit within the transverse width of the spinal column and achieve more substantial engagement into each adjacent vertebral body.

As shown in FIG. 4, bone dowel 100 is shown having openings 104 passing therethrough to communicate with passageway 106. Passageway 106 may be filled with bone material or any natural or artificial bone-growth material or fusion promoting material such that bone growth occurs from one vertebral body to another vertebral body through openings 104 to the material within passageway 106. While openings 104 have been shown in the drawings as being generally oval, it is appreciated that openings 104 and passageway 106 may have any shape, size, or form suitable for use in a bone dowel without departing from the scope of the present invention. Also, the number of openings may be varied or no openings may be present on bone dowel 100.

As shown in FIG. 6, bone dowel 100 has an engagement area at trailing end 114 in the form of two threaded openings 116 for engaging a driver instrument having a removable engagement portion for intimately engaging openings 116. A threaded portion of the driver instrument, which in one embodiment extends as a rod through a hollow tubular member and can be rotationally controlled, screws into threaded openings 116 and binds bone dowel 100 and the driver instrument together. Once affixed to the bone dowel driver instrument, bone dowel 100 may then be introduced through a hollow cylindrical tube and driven into the cylindrical hole that has been drilled across the disc space. The bone dowel driver instrument may then be impacted by a mallet, or similar device, to linearly advance bone dowel 100 across the disc space. Once bone dowel 100 is fully linearly inserted across the disc space, ratchetings 108 engage the bone of the adjacent vertebral bodies, and the bone dowel driver instrument is detached from bone dowel 100. A procedure for drilling the holes across the disc space and instrumentation pertaining thereto are described in applicant's U.S. Pat. No. 5,484,437, issued Jan. 16, 1996, incorporated herein by reference.

While FIG. 6 shows the engagement area at trailing end 114 with two openings 116 with interior threads for engagement with a driver instrument, alternative trailing end embodiments include: a single threaded opening, and more particularly an opening that is along the longitudinal axis of the dowel; multiple openings with only one opening being threaded while the other was adapted to receive a peg; a slot(s) or keyway(s) across the trailing end with a threaded opening that functions to cooperatively engage and disengage a driver; indentations about the perimeter; or any known and published way for engaging a bone dowel or implant.

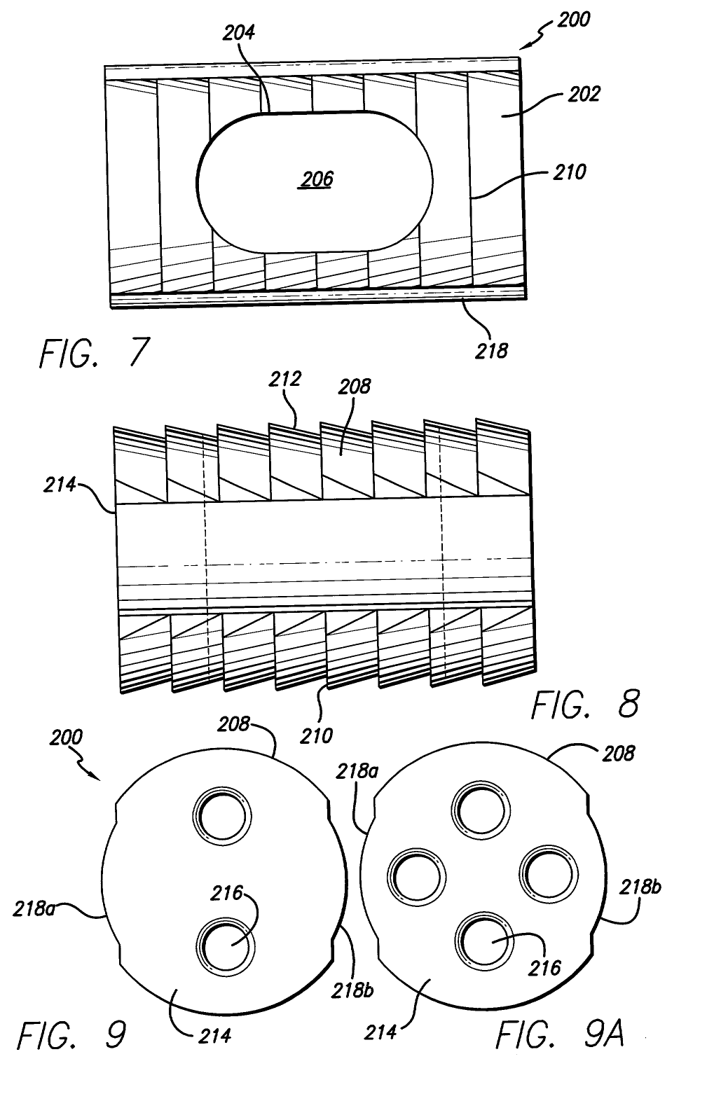

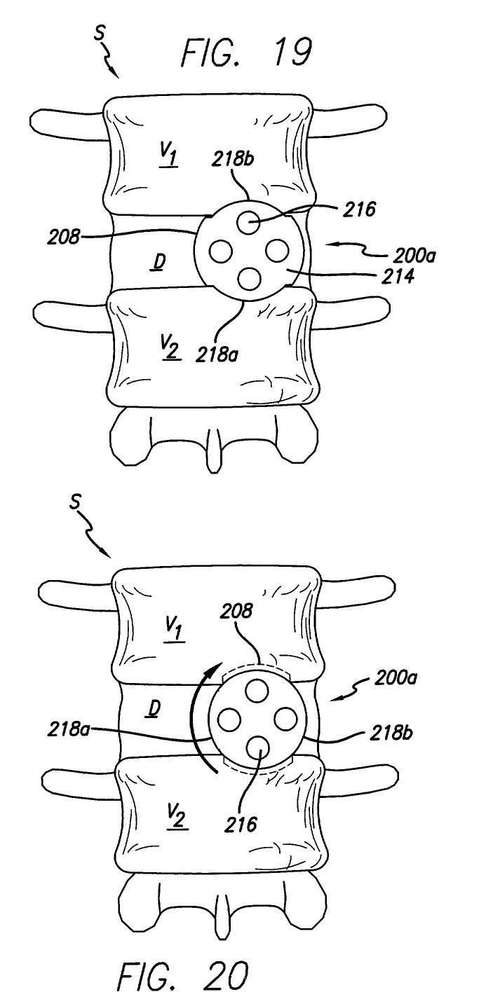

As shown in FIGS. 7-9A, an alternative embodiment of the bone dowel is shown and generally referred to by the number 200. Bone dowel 200 is similar to bone dowel 100 except that ratchetings 208 along at least a portion of the sides are cut off, leaving a round minor root exposed at reduced sides 218a, 218b and ratchetings 208 on approximately the top and bottom quarters as best seen in FIGS. 9 and 9A. FIG. 9A is an alternative embodiment for a trailing end configuration of the bone dowel of FIG. 7. FIG. 9A shows four threaded openings 216 adapted for engaging a driver instrument. This embodiment lends itself easily to an elegant push-in and twist method of insertion as will be described in more detail below and with reference to FIGS. 19 and 20.

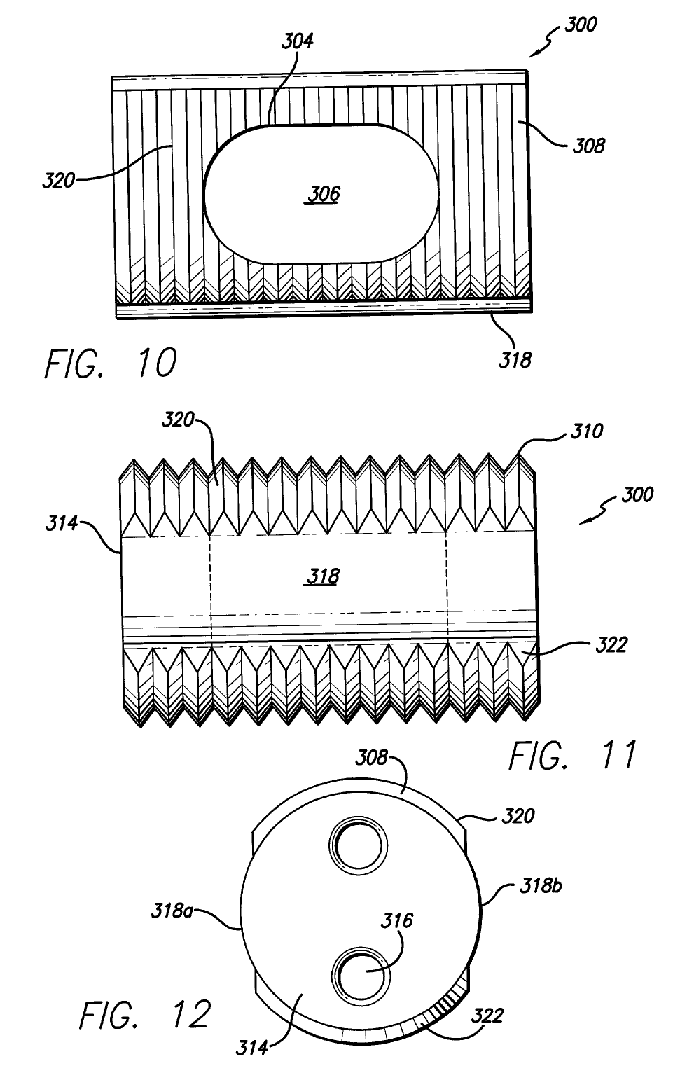

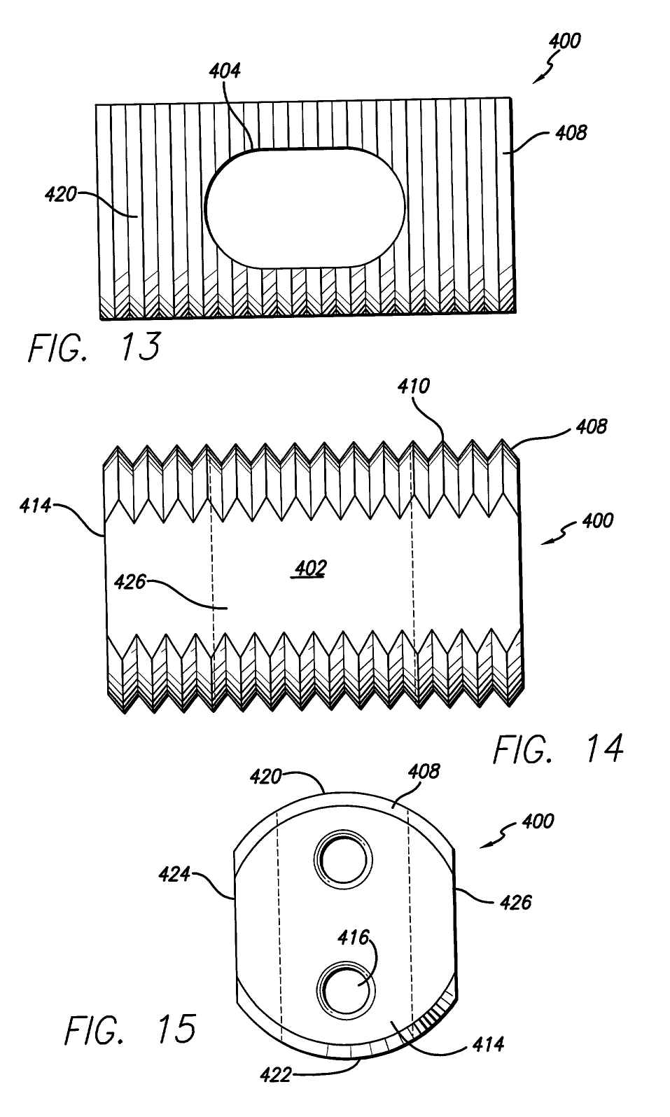

FIGS. 10-12 show yet another alternative embodiment of a bone dowel of the present invention. Bone dowel 300 is similar to bone dowel 200 except that ratchetings 308 are in a saw-tooth configuration rather than the forward-facing configuration of implants 100 and 200. The saw-tooth configuration of ratchetings 308 promotes greater stability in both directions along the axis of insertion of bone dowel 300. The saw-tooth configuration is especially suited for the push-in and turn method of insertion to be described in greater detail below.

As shown in FIGS. 13-15, an alternative embodiment of the bone dowel of the present invention is shown and is generally referred to by the numeral 400. Bone dowel 400 has a similar configuration to that of bone dowel 200, except that it comprises a partially cylindrical member having arcuate portions 420 and 422 which are arcs of the same circle with portions of its outer wall 402 that are flattened so as to present first and second flat sides 424, 426.

As shown in FIG. 13, bone dowel 400 has a major diameter equal to the distance between two diametrically opposite, non-flattened segments, such as arcuate portions 420 and 422 that are arcs of the same circle. The width of bone dowel 400 is equal to the distance between a flattened segment and a point diametrically opposite the flattened segment, such as the distance between first and second flat sides 424, 426.

Bone dowel 400 is preferably used with a second, identical bone dowel 400 such that both bone dowels are implanted across the disc space with the flat side of one bone dowel facing and lying adjacent to the flat side of the second bone dowel. When implanted, the combined overall width of the two bone dowels is less than twice the maximum diameter of the bone dowels. Bone dowels 400 are inserted by linear advancement as described above for bone dowel 100.

As shown in FIG. 14, ratchetings 408 are shown in the saw-tooth configuration, which affords the bone dowel greater stability within the disc space.

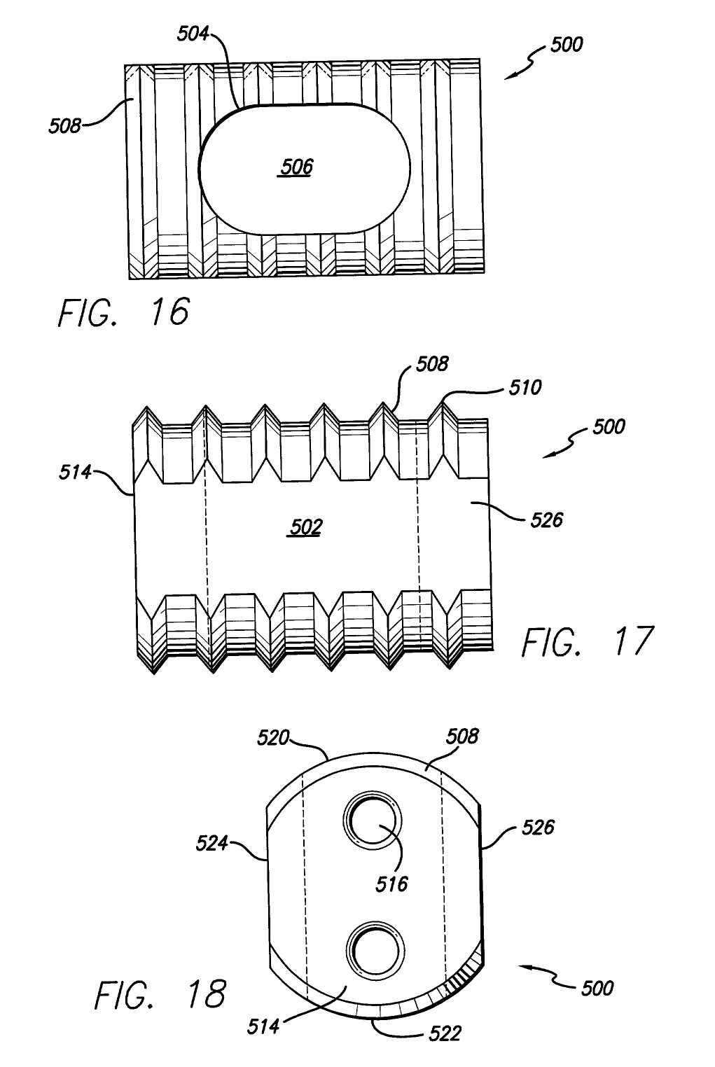

As shown in FIG. 15, the effect of having first and second flat sides 424, 426 is that the overall width of bone dowel 400 is substantially reduced while the height of bone dowel 400 remains the maximum diameter of the cylindrical portion of bone dowel 400.

It is appreciated that it is also within the scope of the present invention that bone dowel 400 could have only one flat side. This configuration is appropriate, where the width of bone dowel 400 need only be slightly reduced with respect to its maximum diameter, to prevent the combined overall width of two such bone dowels from exceeding the transverse width of the spinal column.

As shown in FIG. 15, when viewed on end, bone dowel 400 of the present invention has externally the geometrical configuration of a circle with a portion of each side tangentially amputated vertically to form the first and second flat sides 424, 426.

FIGS. 16-18 show an alternative embodiment of the bone dowel of FIGS. 13-15. In FIGS. 16 and 17, rachetings 508 are shown as a spaced-apart saw-tooth configuration. Such a configuration reduces resistance encountered during placement of the bone dowel while still retaining a measure of stability within the disc space.

As shown in FIGS. 19 and 20, one of two bone dowels 200a is shown inserted across the disc space D. Implanting two bone dowels of the present invention side-by-side will create a construct having a decreased overall combined width when compared to two threaded bone dowels placed side-by-side. The decreased combined overall width of the two bone dowels is the difference between the root and major diameters of the bone dowels and is achieved by utilizing surface roughenings, such as ratchetings 208 for stability. The surface roughenings allow the two bone dowels to come into considerably closer approximation to one another and require less total transverse width for their insertion than is possible for two threaded, cylindrical, bone dowels having identical root diameters because of the requisite thread height of such threaded bone dowels. Reducing the offset between bone dowels allows for the use of larger diameter bone dowels which can then still fit within the transverse width of spinal column S and achieve more substantial engagement into the adjacent vertebral bodies V₁, V₂.

Prior to implantation, two partially overlapping cylindrical holes are drilled across disc space D and into adjacent vertebral bodies V₁, V₂. The holes are drilled sufficiently overlapping to allow two bone dowels 200a and 200b (not shown) to be implanted with the reduced sides 218a, 218b being generally perpendicular to the plane of disc space D, disc space D being in a plane perpendicular to the longitudinal vertical axis of spinal column S as shown in FIG. 19.

Bone dowels 200a and 200b may be inserted separately such that once a first bone dowel 200a is fully linearly inserted across disc space D, a second bone dowel 200b is driven across disc space D, so that reduced sides 218a or 218b of each bone dowel are adjacent to each other and preferably are touching. In this manner, the two bone dowels are implanted across disc space D and engage the bone of adjacent vertebral bodies V₁, V₂ without exceeding the transverse width of spinal column S. Before implanting the second bone dowel, bone dowel 200a is rotated approximately 90 degrees, such that ratchetings 208 engage each vertebral body V₁, V₂ to secure the bone dowel into position. Alternatively, bone dowels 200a, 200b may be implanted across disc space D simultaneously by placing them adjacent to one another with the reduced sides facing each other, in the orientation described above, prior to implantation. The two bone dowels are then linearly advanced into the drilled holes across disc space D. Thus, the surgeon has the option of performing separate push-in and twist insertions, or a single simultaneous insertion of the two bone dowels. It is appreciated that there are other methods of inserting bone dowels that come within the broad scope of the present invention.

Referring again to FIGS. 19 and 20, as the height of each bone dowel is sufficient to cross disc space D and into each adjacent vertebral body V₁, V₂, each bone dowel engages the bone of the adjacent vertebral body while the combined width of the two bone dowels does not exceed the transverse width of spinal column S. As a result, the advantages of placing two cylindrical bone dowels side-by-side across disc space D may be obtained without exceeding the width of spinal column S.

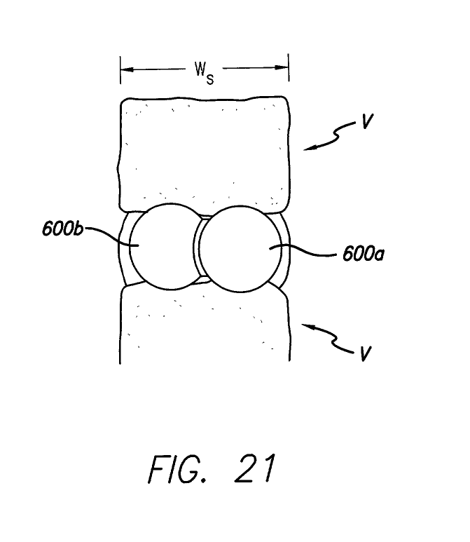

It should be appreciated that many variations of the present inventive concept are possible and come within the broad scope of the present invention. For example, although generally cylindrical bone dowels have been described, the bone dowel of the present invention may exist as other shapes (from a cross-sectional view) such as a saucer, ellipse, or crescent, just to name a few. As shown in FIG. 21, a crescent-shaped bone dowel 600b may be fabricated using known techniques to combine with a generally cylindrical bone dowel 600a and thus widen the width of the overall construct to a lesser extent than using two cylindrical bone dowels placed side-by-side.

The present bone dowel may be tapered in order to best suit the needs of the surgeon and patient, for example, using convergent walls to restore lordosis in the lumbar regions of the spine.

The bone dowel of the present invention may have a plurality of passageways or channels. The size of the bone dowel of the present invention generally has an overall length in the range of 20 mm to 30 mm, with 25 mm being preferred, and a maximum diameter in the range of 14 mm to 24 mm, with 18 mm being preferred when inserted in the lumbar spine from the posterior approach, and 20 mm being preferred when inserted in the lumbar spine from the anterior approach. The bone dowel of the present invention is quite appropriate for use in the cervical and thoracic spine as well. In the cervical spine, such bone dowels would have a length in the range of 10-18 mm, with 12 mm being preferred and a maximum diameter in the range of 12-20 mm, with the preferred diameter being 16 mm. In the thoracic spine, such bone dowels would have a length in the range of 16-26 mm and a diameter in the range of 14-20 mm, with the preferred diameter being 16 mm. In addition to the foregoing dimensions, the bone dowel of the present invention preferably has a width for use in the cervical spine in the range of 8-16 mm, with the more preferred width being 10-14 mm; for use in the lumbar spine in the range of 18-26 mm, with the more preferred width being 18-20 mm; and for use in the lumbar spine in the range of 18-26 mm, with the more preferred width being 20-24 mm.

Each bone dowel of the present invention may or may not include one or more openings in the surface to promote fusion. The size and quantity of the openings will vary depending upon their intended purpose. The shape of the openings may be, for example only, ovals, slots, grooves, and circles, or the naturally occurring shape of the canal through the bone so long as to satisfactorily allow fusion to occur. The bone dowel of the present invention is preferably completely composed of cortical bone since cortical bone provides a superior fusion-enhancing surface. However, it is also to be appreciated that different combinations of cortical bone and of one or more other materials suitable for human implantation may be used.

The trailing end of each bone dowel is preferably anatomically configured to utilize the apophyseal rim bone around the perimeter of each vertebral body to help support the bone dowels. Examples of such configurations are in applicant's co-pending U.S. application Ser. No. 09/263,266, filed Mar. 5, 1999, and entitled “Implant with Anatomically Conformed Trailing End,” the disclosure of which is hereby incorporated by reference.

The passageway is preferably adapted to hold any natural or artificial osteoconductive, osteoinductive, osteogenic, or other fusion enhancing material. Some examples of such materials are bone harvested from the patient, or bone growth-inducing material, such as, but not limited to, hydroxyapatite, hydroxyapatite tricalcium phosphate, genes coding for production of bone, or bone morphogenetic protein. The bone dowel of the present invention may be filled and/or coated with a bone ingrowth inducing material, such as, but not limited to, hydroxyapatite or hydroxyapatite tricalcium phosphate or any other osteoconductive, osteoinductive, osteogenic, or other fusion enhancing material.

The bone dowel of the present invention may also be adapted to receive opposed, vertebral body engaging screws of cortical bone, bioresorbable material, or other material suitable for human implantation through its trailing end. Examples of such screws are in applicant's co-pending U.S. application Ser. No. 09/556,055, filed May 5, 2000, entitled “Screws of Cortical Bone and Method of Manufacture Thereof,” the disclosure of which is hereby incorporated by reference.

The bone dowel of the present invention may include surface roughenings. Surface roughenings enhance the stability of the bone dowel and resist dislodgement once the bone dowel is implanted across the disc space. Other examples of surface roughenings include holes, grooves, knurling, slots, projections, and the like. Ratchetings are the preferred form of surface roughenings. The ratchetings may come in many forms, for example only, forward facing (FIG. 4), saw-tooth (FIG. 11), and spaced saw-tooth (FIG. 17). Many other combinations are possible and also within the broad scope of the present invention. Forward-facing ratchetings provide for a “one-way” insertion of the bone dowel as the movement of the bone dowel in the opposite way is inhibited by the engagement or the engaging edges with each vertebral body. Saw-tooth and spaced saw-tooth ratchetings provide an enhanced stability in either direction along the insertion axis of the bone dowel.

While the present invention has been described in detail with regard to the preferred embodiments, it is appreciated that other variations of the present invention may be devised which do not depart from the inventive concept and scope of the present invention.

What is claimed is:

1. An interbody spinal implant for insertion at least in part into an implantation space formed across a disc space between adjacent vertebral bodies of a human spine and into at least a portion of the endplates of the vertebral bodies, said implant comprising:
a body having a leading end for insertion first into the disc space and a trailing end opposite said leading end;
opposite upper and lower surfaces adapted to be placed in contact with and to support the adjacent vertebral bodies, said upper and lower surfaces being arcuate;
an opening passing through said upper and lower surfaces for permitting for the growth of bone from adjacent vertebral body to adjacent vertebral body through said implant; and
said implant being manufactured from a composite of cortical bone and at least one bioresorbable material, said cortical bone and said at least one bioresorbable material being combined to form a machinable material from which said implant is manufactured.

2. The implant of claim 1, wherein said composite includes cortical bone fibers.

3. The implant of claim 1, wherein said composite includes cortical bone filaments.

4. The implant of claim 1, wherein said composite includes cortical bone particles.

5. The implant of claim 1, wherein said bioresorbable material includes plastics.

6. The implant of claim 1, wherein said bioresorbable material includes ceramic.

7. The implant of claim 1, wherein said bioresorbable material includes composite plastics.

8. The implant of claim 1, further comprising at least one protrusion extending from at least one of said upper and lower surfaces for engaging at least one of the adjacent vertebral bodies to maintain said implant within the implantation space.

9. The implant of claim 8, wherein said protrusion comprises at least one of a ridge, ratcheting, spline, and knurling.

10. The implant of claim 1, wherein said upper and lower surfaces are porous.

11. The implant of claim 1, wherein said upper and lower surfaces include a bone ingrowth surface.

12. The implant of claim 1, wherein at least a portion of said upper and lower surfaces are in an angular relationship to each other from trailing end to leading end for allowing angulation of the adjacent vertebral bodies relative to each other.

13. The implant of claim 1, wherein at least a portion of said leading end is tapered for facilitating insertion of said implant between the two adjacent vertebral bodies.

14. The implant of claim 1, in combination with a fusion promoting material other than bone.

15. The implant of claim 1, further in combination with bone morphogenetic protein.

16. The implant of claim 1, further in combination with genetic material coding for production of bone.

17. The implant of claim 1, further in combination with a chemical substance to inhibit scar formation.
